(19) United States
(12) Patent Application Publication
(10) Pub. No.: US 2024/0013881 A1
(43) Pub. Date: Jan. 11, 2024

(54) METHODS FOR AUTOMATED PROVISION OF PATIENT WITH ELECTRONIC PRESCRIPTION AND MEDICINES BY MEANS OF INFORMATION SYSTEM OF ELECTRONIC PRESCRIPTIONS

(71) Applicant: MILI HEALTHCARE TRADE DMCC, LIMITED LIABILITY COMPANY, Dubai (AE)

(72) Inventor: Oleksii Lytvynenko, Kyiv (UA)

(21) Appl. No.: 18/253,165

(22) PCT Filed: Nov. 15, 2021
(86) PCT No.: PCT/UA2021/000093

§ 371 Date: May 16, 2023

(30) Foreign Application Priority Data
Nov. 16, 2020 (UA) A 2020 07320

(51) Int. Cl. G16H 20/13; G16H 10/60; G16H 80/00; G06Q 20/38; G06Q 20/40; G06Q 20/20; H04L 9/32

(52) U.S. Cl. CPC G16H 20/13; G16H 10/60; G16H 80/00; G06Q 20/3829; G06Q 20/3825; G06Q 20/4014; G06Q 20/38215; G06Q 20/202; H04L 9/321; G06Q 2220/00

(57) ABSTRACT

The invention relates to the field of medicine, namely to a set of actions, technologies and measures used in provision of medical care by means of remote means for remote provision of patients with prescription medicines by means of the information system of electronic prescriptions. The method of automated provision of a patient with an electronic prescription for prescription medicines includes obtaining a unique electronic prescription identifier with prescribed medicines by a patient, which according to the methods of automated delivery of prescription medicines to a patient by means of a unique electronic prescription identifier includes obtaining the prescribed medicines by a patient personally in the points of sale of medicines or by remote delivery. The technical result of the proposed invention is to develop methods for provision of a patient with an electronic prescription and prescription medicines by means of the information system of electronic prescriptions that implement the possibility to verify originality of each individual medicine in the order and the entire order upon its receipt by means of the server of validation of security elements, error-free identification of a person and elimination of abuse and attempts of identity substitution during identification by means of convenient process of identification and authentication at all stages, different levels of control at the stages of prescribing and dispensing for certain groups of medicines, use of different levels of identification and authentication of the patient during dispensing of medicines, by means of standard and multi-factor authentication, control over circulation of medicines at each stage of their movement at the level of each individual primary package of the medicine by means of the goods movement recording service, control over authenticity of origin of the medicines at each stage of their movement by means of the server of validation of security elements.

### FIELD OF THE INVENTION

The invention relates to the field of medicine, namely to a set of actions, technologies and measures used in provision of medical care using remote means for remote provision of patients with prescription medicines by means of the information system of electronic prescriptions.

### BACKGROUND OF THE INVENTION

The prior art provides the prescription drugs selling method based on a two-dimensional code (Chinese application No. CN 105046542 A, Nov. 11, 2015) where the application describes the method for selling medicines based on use of a two-dimensional code, according to which the doctor by means of his/her electronic terminal enters information about the prescription and the patient for whom the prescription is issued into the service platform, encodes this data by means of a QR code and provides the patient, at the point of sale the patient provides a QR code with the prescription, the data are sent to the service platform to verify their validity, the service platform verifies compliance of the prescription and the patient with the data of the doctor who issued the prescription, and in case of successful data verification the medicine is sold to the patient and the corresponding entry is made in the service system.

The disadvantages of the known method for selling medicines are inability to order medicines remotely, inability to control circulation of medicines and inability to verify authenticity of origin of medicines sold at points of sale.

The prior art provides the system of electronic prescription exchange “Pharmtaxi” (https://www.pharmtaxi.com/), in which prescriptions are transmitted from a doctor to patient and from a patient to a pharmacy, wherein the doctor prescribes treatment, generates an electronic prescription and sends it to the patient, the patient receives the prescription on an electronic device, for example, in the form of a QR code, or can take a picture thereof and send the same to the nearest pharmacy, in the pharmacy the patient presents the prescription, pays and receives the order, the system also has the possibility to verify medicines for compatibility and authenticity of origin.

The disadvantages of the known system are inability to identify and authenticate a patient, inability to control circulation of medicines dispensed to a patient at the point of sale, absence of control at the stages of prescribing and dispensing for certain groups of medicines and inability to use different levels of identification and authentication during dispensing of medicines.

The prior art provides a computer-based prescription system for medicaments (US application No. US 2015254423 A1, Oct. 9, 2015), which describes generation of a doctor's electronic prescription signed with the use of a digital certificate, sending a signed prescription to a mobile device of a user-patient (for example, via NFC), reading an electronic prescription from a mobile device at a supplier of medicines for their purchase. This uses encryption by means of a pair of private and public keys.

The disadvantages of the known technical solution are absence of control at the stages of prescribing and dispensing for certain groups of medicines, absence of implementation of multi-factor authentication of the patient for certain groups of prescription medicines, such as medicines with special control over circulation, absence of full control over circulation of medicines from the moment of issuance of electronic prescription to the moment of receipt of medicines by a patient.

The prior art provides the remote ordering terminal for prescription and over-the-counter medications (Australian patent No. AU 2011264382 B2, Feb. 3, 2017), by means of which the medicines available only on prescription or over-the-counter medicines are ordered by mail from pharmacies or other distributors, wherein the doctor's electronic prescription is sent to the patient in any suitable way, then verified by an automated system for the fact that it is prescribed by a particular doctor to a particular patient-presenter, and a unique bar code is used to encode the doctor's prescription.

The disadvantages of the known technical solution are insufficiently reliable system of identification and authentication of the patient, insufficient protection of the doctor's prescription, absence of full control over circulation of medicines by the system that may lead to sale of counterfeit medicines or receipt of medicines under forged doctor's prescription, as well as absence of control over authenticity of origin of medicines.

The prior art provides the secure method for delivering regulated items including drugs to a patient (US application No. US 20110245967 A1, Jun. 12, 2011) from which it is known to enter the information on the doctor who has the right to issue prescriptions into the database, to enter the information on the patient (his anthropometric data, data on medicines, etc.) into the database, analysis of the prescription issued for the particular patient and comparison and reconciliation of the prescription with the information on the patient available in the database.

The disadvantages of this method for delivery are absence of verification of authenticity of origin of each individual medicine in the order and the entire order upon its receipt from the delivery service, absence of implementation of multi-factor authentication of the patient, inability of control over circulation of medicines delivered to the patient at the point of sale, and absence of different levels of control at the stages of prescribing and dispensing for certain groups of medicines, in particular, medicines with special control over circulation.

### SUMMARY OF THE INVENTION

The aim of the invention is to provide the methods for provision of patients with electronic prescription and prescription medicines and the information system of electronic prescriptions for implementing methods that would provide convenient remote ordering of medicines, including immunobiological medicinal products, medical equipment, medical products, narcotic drugs, psychotropic substances and their precursors, convenient authentication of the patient that allows for error-free identification of a person eliminating abuse and attempts to change identity during identification, different levels of control at the stages of electronic prescribing and dispensing for certain groups of medicines, as well as use of different levels of identification and authentication of the patient during dispensing of medicines, with different levels of control over circulation and timely detection of emerging of counterfeit medicines in the chain from manufacturer/importer to final consumer-patient.

The technical result of the present invention is to provide the methods for provision of patients with electronic prescription and prescription medicines and the information system of electronic prescriptions for implementing methods that implement possibility to verify authenticity of origin of each individual medicine in the order and the entire order upon its receipt by means of the server of validation of security elements, error-free identification and elimination of abuse and attempts of identity spoofing during identification by means of convenient process of identification and authentication at all stages, different levels of control at the stages of prescribing and dispensing for certain groups of medicines, use of different levels of identification and authentication of the patient during dispensing of medicines, by means of standard and multi-factor authentication of the patient, control over circulation of medicines at each stage of their movement at the level of each individual primary package of the medicine by means of the goods movement recording service, control over originality of medicines at each stage of their movement by means of the server of validation of security elements.

In addition, the present invention provides improvement of quality of medical care, promotion of modern digital technologies in the medical system, collection and transmission of complete data for current and future electronic registers, budget savings by means of full automation of the entire process, control over circulation of medicines, audit, easy introduction of the system by virtue of the fact that the system is a single solution, automated data exchange with all market participants and introduction of digital technologies in the business processes of the institution.

### BRIEF DESCRIPTION OF THE DRAWINGS

The present invention is illustrated by the drawings:

FIG. 1 shows a block diagram of the information system of electronic prescriptions that illustrates passage of the technological stages of the proposed methods.

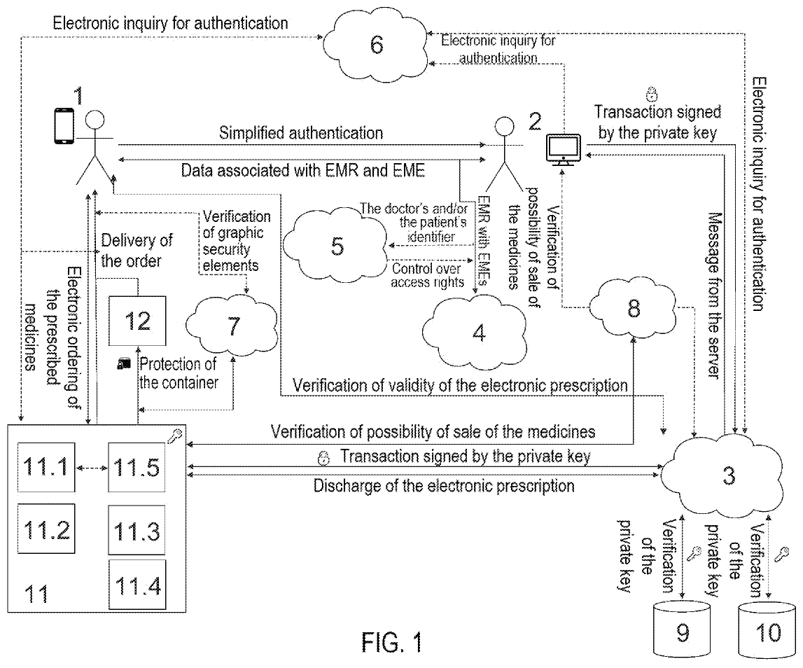

FIG. 2 shows an embodiment of the invention, namely a method for automated provision of a patient in a medical institution with prescription medicines.

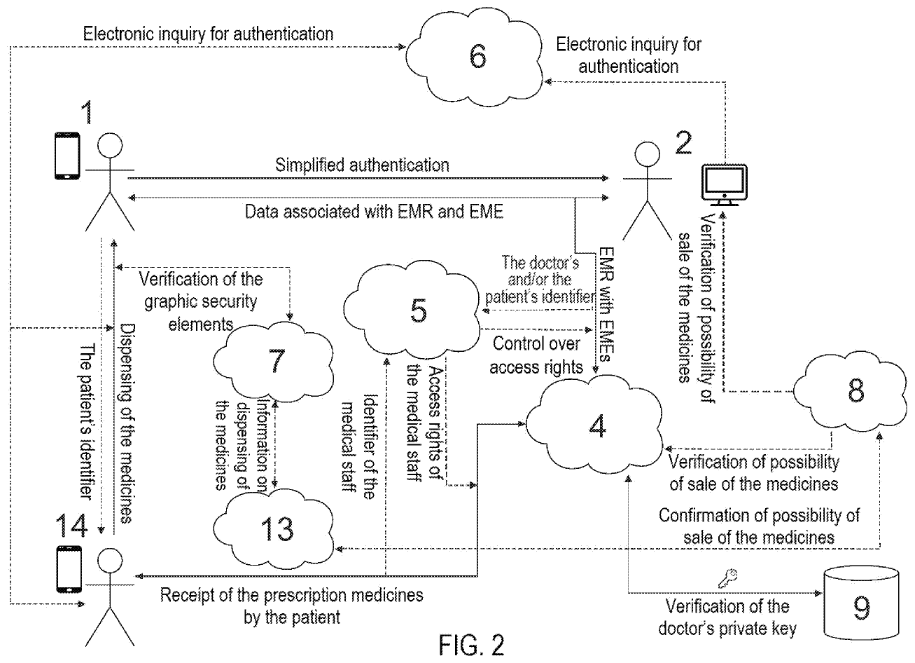

### DETAILED DESCRIPTION OF THE INVENTION

The object is achieved by virtue of the fact that the proposed method of automated provision of a patient with an electronic prescription for prescription medicines includes stages where:
by means of the doctor's work terminal 2, simplified authentication of the patient is performed, during this authentication the patient's identifier and/or the patient's public key and the set of data associated with the identifier of the patient's electronic medical record (EMR) stored on the server of electronic medical entries 4 are obtained,
the medicines are prescribed to the patient by means of the doctor's work terminal 2, wherein
   if the prescribed medicines include at least one prescription medicine, then, by means of the patient identification and authentication service 6, standard authentication of the patient, including at least one unique authentication method, is additionally performed, for which purpose at least one electronic inquiry is made from the doctor's work terminal 2 to the patient identification and authentication service 6 to confirm the identity of the patient, and
   if the prescribed medicines include at least one prescription medicine with special control over circulation, then multi-factor authentication of the patient, including at least two unique authentication methods, is additionally performed, for which purpose at least two electronic inquiries are made from the doctor's work terminal 2 to the patient identification and authentication service 6 to confirm the identity of the patient,
a transaction is generated at the doctor's work terminal 2 to the server of electronic prescriptions 3, the transaction including the doctor's identifier and/or the doctor's public key, the patient's identifier and/or the patient's public key, the set of data associated with the prescribed medicines, the set of data associated with the date/time of generation of the transaction and the set of data associated with validity period of the electronic prescription, and the generated transaction is signed with at least the doctor's private key, wherein
   electronic medical entry (EME) with prescribed medicines is made in the patient's EMR, for which purpose an electronic inquiry signed by the doctor's private key is sent from the doctor's work terminal 2 to the data access authorization service 5, the electronic inquiry including the doctor's identifier and/or the doctor's public key, the patient's identifier and/or the patient's public key and the set of data associated with the patient's EMR identifier, and in case of successful authorization the permission to make changes to the patient's EMR on the server of electronic medical entries 4 is granted and EME associated with the prescribed medicines is made in the patient's EMR,
the transaction signed by the doctor's private key is received on the server of electronic prescriptions 3 and the doctor's accreditation is verified by sending an inquiry for the doctor's public key to the database of accredited doctors 9 and verifying thereby validity of the signature of the transaction, and
in case of the positive verification of the doctor's accreditation, the electronic prescription is saved on the server of electronic prescriptions 3, after that
a message of generation of the electronic prescription including a unique electronic prescription identifier and its term is received from the server of electronic prescriptions 3 and it is sent to the patient's terminal 1.

Also, the object is achieved by virtue of the fact that the proposed method for automated receipt of prescription medicines by means of a unique electronic prescription identifier includes the stages where:
an electronic order of the prescribed medicines is generated at the point of sale by entering a unique electronic prescription identifier into the terminal of the point of sale 11,
by means of the terminal of the point of sale 11, a transaction is generated to the server of electronic prescriptions 3, the transaction including a unique electronic prescription identifier, the patient's identifier and/or the patient's public key, a set of data associated with the prescribed medicines, the identifier of the point of sale and/or the public key of the point of sale, and the generated transaction is signed with at least the private key of the point of sale, wherein
   if the prescribed medicines include at least one prescription medicine, then, by means of the patient identification and authentication service, standard authentication of the patient, including at least one unique authentication method, is additionally performed, for which purpose at least one electronic inquiry is made from the terminal of the point of sale 11 to the patient identification and authentication service 6 to confirm the identity of the patient, and
   if the prescribed medicines include at least one prescription medicine with special control over circulation, then multi-factor authentication of the patient, including at least two unique authentication methods, is additionally performed, for which purpose at least two electronic inquiries are made from the terminal of the point of sale 11 to the patient identification and authentication service 6 to confirm the identity of the patient, after that
possibility of sale of the prescribed medicines is verified, for which purpose an electronic inquiry is made from the terminal of the point of sale 11 to the electronic register of medicines and services 8 and an automated response, including information on whether the prescribed medicines are approved for medical practice and validity term of their registration certificate, is received, and
validity of the transaction signed with at least the private key of the point of sale is verified on the server of electronic prescriptions 3, for which purpose an inquiry for the public key of the point of sale is made to the database of registered points of sale 10, validity period of the electronic prescription and its validity are verified and, in case of the positive result of verifications from the server of electronic prescriptions 3, confirmation of possibility of delivery of the order of the prescribed medicines is sent to the terminal of the point of sale 11, after that
automated dispensing of the ordered medicines to the patient is performed and, if necessary, by means of the patient's terminal 1, authenticity of origin of the medicines is verified by verifying their graphic security elements on the server of validation of security elements 7, wherein
   by means of the terminal of the point of sale 11, an electronic inquiry for at least partial discharge of the electronic prescription is sent to the server of electronic prescriptions 3, and the information on dispensing of the prescribed medicines is transmitted to the goods movement recording service 13.

Also, the object is achieved by virtue of the fact that the proposed method for automated delivery of prescription medicines to the patient by means of a unique electronic prescription identifier includes the stages where:
an electronic order of prescribed medicines is generated at the patient's terminal 1, the electronic order including a unique electronic prescription identifier, the patient's identifier and/or the patient's public key, the set of data associated with the prescribed medicines, after that the electronic order is signed with the patient's private key and transmitted via the communication channels to the terminal of the point of sale 11,
the electronic order signed with the patient's private key is received by means of the terminal of the point of sale 11 and a transaction is generated to the server of electronic prescriptions 3, the transaction including a unique electronic prescription identifier, the patient's identifier and/or the patient's public key, the set of data associated with the prescribed medicines, the identifier of the point of sale and/or the public key of the point of sale and the generated transaction is signed with at least the private key of the point of sale, wherein
   if the prescribed medicines include at least one prescription medicine, then, by means of the patient identification and authentication service 6, standard authentication of the patient, including at least one unique authentication method, is additionally performed, for which purpose at least one electronic inquiry is made from the terminal of the point of sale 11 to the patient identification and authentication service 6 to confirm the identity of the patient, and
   if the prescribed medicines include at least one prescription medicine with special control over circulation, then multi-factor authentication of the patient, including at least two unique authentication methods, is additionally performed, for which purpose at least two electronic inquiries are made from the terminal of the point of sale 11 to the patient identification and authentication service 6 to confirm the identity of the patient, after that
possibility of sale of the prescribed medicines is verified, for which purpose an electronic inquiry is made from the terminal of the point of sale 11 to the electronic register of medicines and services 8 and an automated response, including information on whether the prescribed medicines are approved for medical practice and validity term of their registration certificate, is received, and
validity of the transaction signed with at least the private key of the point of sale is verified on the server of electronic prescriptions 3, for which purpose an inquiry for the public key of the point of sale is made to the database of registered points of sale 10, validity period of the electronic prescription and its validity are verified and, in case of the positive result of verifications from the server of electronic prescriptions 3, confirmation of possibility of delivery of the order of the prescribed medicines is sent to the terminal of the point of sale 11, after that
the prescribed medicines are placed in a container with opening control protected by a separate security element, wherein the container with opening control is configured to ensure verification of the graphic security elements of individual medicines therein without opening the container, the information on dispensing of the prescribed medicines is transmitted to the goods movement recording service 13 and automated dispensing of the prescribed medicines is performed to the delivery service in the container with opening control and the container with the prescribed medicines is delivered to the patient, after that identification and authentication of the patient is performed by means of the terminal of the delivery service 12, wherein
   if the prescribed medicines include at least one prescription medicine, then, by means of the patient identification and authentication service 6, standard authentication of the patient, including at least one unique authentication method, is additionally performed, for which purpose at least one electronic inquiry is made from the work terminal of the delivery service 12 to the patient identification and authentication service 6 to confirm the identity of the patient, and
   if the prescribed medicines include at least one prescription medicine with special control over circulation, then multi-factor authentication of the patient, including at least two unique authentication methods, is additionally performed, for which purpose at least two electronic inquiries are made from the work terminal of the delivery service 12 to the patient identification and authentication service 6 to confirm the identity of the patient,
if necessary, by means of the patient's terminal 1, integrity of the separate security element of the container and authenticity of origin of the medicines are verified by verifying their graphic security elements on the server of validation of security elements 7, wherein verification of medicines is performed without opening the container and breaking the separate security element of the container, and, in case of the positive result of verification, the order is delivered to the patient, after that
by means of the terminal of the point of sale 11, an electronic inquiry for at least partial discharge of the electronic prescription is sent to the server of electronic prescriptions 3 and the information on dispensing of the prescribed medicines is transmitted to the goods movement recording service 13, and
in case of breaked separate security element of the container or negative result of verification of authenticity of origin of the medicines, the container with medicines is returned to the point of sale.

In addition, the object is achieved by virtue of the fact that the information system of electronic prescriptions for implementation of the methods includes
the server of electronic prescriptions 3, including the database of accredited doctors 9 and the database of points of sale 10, configured to process electronic transactions, store electronic prescriptions, verify accreditation of the doctors and verify the registered points of sale,
the server of electronic medical entries 4 configured to store the patient's EMR, including a plurality of EME, and to prove access thereto,
the data access authorization service 5 configured to control the data access rights to the data of the server of electronic medical entries 4, wherein the data include EMR with all EMEs,
the electronic register of medicines and services 8, including the database providing storage and processing of information associated with medicines, medical devices and services, configured to verify the possibility of sale of the prescribed medicines,
the patient identification and authentication service 6 configured to identify and authenticate the patient by the patient's identifier and verify authenticity of the identification data provided by the patient, wherein the identification and authentication service is specially adapted to perform standard and multi-factor authentication of the patients,
the server of validation of security elements 7 configured to be accessible by the patient's terminal 1 and verify the graphic security elements and the separate security element,
the doctor's work terminal 2 configured to generate electronic transactions, send electronic inquiries and sign them with the doctor's private key, wherein the doctor's work terminal 2 is specially adapted to create an EMR on the server of electronic medical entries 4 and enter/edit EME therein,
the patient's terminal 1 configured to access the patient identification and authentication service 6 and perform standard and multi-factor authentication of the patient, and access to the server of validation of security elements 7 to verify the graphic security elements,
the electronic terminal of the point of sale 11 including a communication module and a means of reading for a unique electronic prescription identifier, wherein the electronic terminal of the point of sale 11 is configured to initiate standard and/or multi-factor authentication of the patient and dispense the prescribed medicines to the patient, and access the server of electronic prescriptions,
the terminal of the delivery service 12 configured to initiate the procedure of identification and authentication of the patient,
   wherein the patient identification and authentication service 6, the electronic register of medicines and services 8, the doctor's work terminal 2, the electronic terminal of the point of sale 11 are connected to the server of electronic prescriptions 3, the data access authorization service 5 is connected to the server of electronic medical entries 4.

According to the present invention, a patient is a person who receives medical care, undergoes medical observation and/or treatment of a disease, pathological state or disorder of health or vital activity, and also receives medical services regardless of whether he has a disease. In order to fully interact with the medical information system, i.e. to obtain an electronic prescription, order prescription medicines, verify authenticity of origin of medicines, etc., the patient must have a patient's terminal. The patient's terminal 1 is a hardware in the form of a mobile phone, smartphone, tablet, laptop, personal computer or other communication device that does not require specialized software, so that the patient, even with a regular mobile phone, is able to use the services of the information system of electronic prescriptions according to the proposed methods. A smart device, such as a smartphone or tablet, can have a specialized software application installed for quick and easy use.

A doctor is a specialist with education who conducts medical observation and/or treatment of a patient, provides him with medical services, prescribes medicines and issues an electronic prescription, etc. Each doctor must be accredited with obtaining a doctor's identifier and the information on the accreditation of doctors is entered into the database of accredited doctors.

A medical staff is a specialist who conducts medical treatment of a patient in a medical institution or during treatment at home as prescribed by a doctor, dispenses and ensures administration of medicines and provision of services, performs medical manipulations, etc.

An electronic prescription is an electronic document on the doctor's prescription of a medicine (active substance or international non-proprietary name (INN) of a medicine), dosage and method of administration. The electronic prescription is stored on the server of electronic prescriptions 3 and allows the patient to purchase and properly use the medicines listed therein. The electronic prescription has a unique electronic prescription identifier by which it can be obtained by the patient and/or by which the electronic prescription can be accessed on the server of electronic prescriptions 3. The unique electronic prescription identifier can be a sequence of characters or a QR code or barcode, or a combination thereof. For example, it can be a barcode with a sequence of numbers below such a code.

A transaction in the present invention is a generated and transmitted data structure containing information on a particular operation. The transaction can include identifiers of a doctor, patient, point of sale, data associated with the prescribed medicines, and other records. The transaction is signed with at least one private key of the initiator of the transaction and can be additionally signed by the private keys of other authorized persons.

A server of electronic prescriptions 3 is a dedicated or specialized remote computer specially adapted to execute service software that provides storage of electronic prescriptions, processing of information related to issuance of electronic prescriptions and fulfillment of orders related to electronic prescriptions and control over the validity period of the electronic prescription.

A server of electronic medical entries 4 is a dedicated or specialized computer for execution of service software that provides storage and processing of information related to electronic medical documentation (electronic medical records) of patients. Access to the server of electronic medical entries 4 is regulated by the data access authorization service 5.

An electronic medical entry (EME) is an electronic entry that contains information on patient's treatment, consultations, diagnostics, manipulations, test results, and other medical operations related to a patient, in particular medical prescriptions, including medicines and doctor's recommendations.

An electronic medical record (EMR) of a patient is a structured record on the server of electronic medical entries 4 comprising the patient's identification data and all its EMEs.

EMR accumulates the entire medical history of the patient and other information about his/her health. The patient's EMR can be accessed by the doctor, medical staff or the patient himself.

A patient identification and authentication service 6 is a hardware and software complex that provides identification of the patient by his/her identifier and verification of authenticity of the identification data provided by the patient (the patient's identity). Possible forms of identification data are: electronic digital signature (EDS), ID-Passport, ID-Card, Mobile-ID, Smart-ID, Bank-ID, FaceID, TouchID, Google-ID, Apple-ID, and identification via social networks, SMS-authentication and other means acceptable for identification of a person.

Simplified authentication of the patient is a method for authentication that does not require verification of authenticity of the identification data provided by the patient (the patient's identity) in third-party services. In particular, simplified authentication can be the reading of any document that contains the patient's personal data, such as a passport, foreign passport, driver's license, etc.

Standard authentication of the patient is authentication that primarily uses a method for authentication of the patient, such as a smart card, USB token, etc. In a preferred embodiment of the invention at least one method for authentication is used for standard authentication of the patient, for example when ordering prescription medicines that do not require special control over circulation.

Multi-factor authentication of the patient is an advanced authentication that uses several unique methods of authentication of the patient simultaneously, i.e. authentication methods that differ from each other. In a preferred embodiment of the invention at least two unique authentication methods are used for multi-factor authentication of the patient. Multi-factor authentication is used, for example, when a patient orders prescription medicines with special control over circulation.

A data access authorization service 5 is a hardware and software complex that provides and verifies access rights to the data stored on the server of electronic medical entries 4, authenticated users: patients, doctors, medical staff, etc. The service 5 is used to create, review, and change EMRs of the patients and introducing EMEs therein by doctors and medical staff.

A server of validation of security elements 7 is a hardware and software complex with use of cloud technologies, which provides verification of authenticity of security elements. This server is used for the patient's verifying the graphic security elements that protect medicines, as well as separate graphic security elements that protect the containers in which the prescribed medicines are delivered to the patient.

An electronic register of medicines and services 8 is a hardware and software complex and/or electronic database that provides storage and processing of information related to medicines, medical devices and services (authorized for sale), as well as a list of international names of medicines (INN) and a list of active substances, the validity term of their registration certificates. The electronic register is used to determine whether the prescribed medicines are approved for use in medical practice and whether the validity term of their registration certificates has not expired. This is an additional mechanism of protection against sale of substandard or counterfeit medicines. Such medicines include, but are not limited to, immunobiological medicinal products, narcotic drugs, psychotropic substances and their precursors, as well as medical devices and medical products.

A database of accredited doctors 9 is a database with an ordered set of structured information or data stored electronically in the information system of electronic prescriptions locally or remotely. The database 9 contains information on accredited doctors and medical staff who have the right to practice medicine, including their public keys, which are used to verify transactions signed with the private keys of doctors or medical staff.

A database of registered points of sale 10 is a database with an ordered set of structured information or data stored electronically in the information system of electronic prescriptions locally or remotely. The database 10 contains information on the points of sale registered in the system that are allowed to sell medicines, including their public keys, which are used to verify transactions signed by the private keys of the points of sale.

A doctor's work terminal 2 is a hardware in the form of a personal computer, laptop, smartphone, tablet computer or other electronic computing equipment that contains specialized software necessary to perform the direct duties of a doctor in the information system of electronic prescriptions. In particular, the doctor's work terminal 2 is used to conduct transactions and initiate the patient's authentication procedure.

A point of sale is a registered point of sale of medicines, equipped with a terminal 11 of the point of sale, such as a vending machine 11.3, electronic trading platform 11.4, pharmacy 11.1, or online pharmacy 11.2, or an accredited distributor of 11.5 of medicines. The point of sale must be equipped with an electronic terminal 11 of the point of sale, connected to the information system of electronic prescriptions or contains a specialized communication module for connection to the system in case of stand-alone points of sale. The electronic trading platform 11.4 in this case is a hardware and software complex of information and technical solutions that combines and classifies information and offers of different points of sale on one resource, provides remote interaction of sellers of medicines and services and patients by means of electronic communication channels. The vending machine 11.3 is a stand-alone device that conducts retail sale of medicines, whose payment and dispensing is performed by means of technical devices that do not require direct human involvement.

In one embodiment, the point of sale can be further equipped with at least one self-service terminal connected to the information system of electronic prescriptions by means of which the patient can verify origin of the medicines received at the point of sale, as well as obtain complete information on the medicine and/or or make an order of medicines himself. The self-service terminals are primarily aimed at patients who do not have smartphones, tablets or other modern means of communication.

A delivery service is an element of the information system of electronic prescriptions that provides delivery of medicines between points of sale and the patient. The delivery service must be equipped with an electronic terminal 12 connected to the information system of electronic prescriptions. In particular, patient authentication, inquiry for discharge of the electronic prescription and transfer of information on dispensing of the prescribed medicines to the goods movement recording service 13 are performed by means of the terminal of the delivery service 12. The work terminal of the delivery service 12 is a hardware in the form of a laptop, smartphone, tablet computer or other mobile electronic computing equipment containing specialized software necessary to perform the direct duties of the delivery service in the information system of electronic prescriptions.

A private key is a unique sequence of characters that forms an electronic digital signature (EDS). The sequence of characters that can be provided electronically can be stored on a file medium, external secured devices (such as USB tokens or smart cards with a chip), on special SIM cards of mobile operators, in the cloud, on chips and other technical solutions of electronic identity cards (for example, ID-passport), etc. that support operation with a private key. Doctors, points of sale and medical staff must be equipped with private keys.

A public key is a key used to verify transactions signed with a private key.

A security element is a unique identifier of a unit of a medicine and/or package of medicines having protection against counterfeiting, duplication, and with opening control that provides its automated or semi-automated reading. It can contain additional information on a medicine in itself or in a separate database. The security element can be implemented in the form of a graphic element, such as a QR code, barcode, DataMatrix, etc., in the form of a holographic element, or a radio frequency element, such as RFID tag and others. In a preferred embodiment, security graphic elements are used.

A goods movement recording service 13 is a hardware and software complex with application of cloud technologies that stores and processes information about changes in the status of the movement and location of goods during their passage in the information system of electronic prescriptions. By virtue of this service, there is a control over movement of medicines and the possibility of substitution and counterfeiting is eliminated.

The information on all movements, returns, seizures and disposal of medicines, as well as dispensing of medicines to the final consumer-patient entered into the goods movement recording service 13. Transfer of information to the goods movement recording service 13 is performed with simultaneous verification by the server of validation of security elements 7, where manufacturers and/or importers provide information on the security elements released for medicines. The information on separate graphic security elements of the containers with the ordered medicines is also transmitted to the server of validation of security elements 7 by the delivery services and/or points of sale. The goods movement recording service 13 monitors the following actions with medicines with special control over circulation (narcotic, psychotropic drugs and their precursors): production, manufacture, purchase, movement, storage, realization (sale, dispensing), importation and exportation in/from the country, use, disposal, etc.

The proposed invention is implemented as follows.

A patient attends a doctor as an appointment or a remote consultation. The doctor performs simplified authentication of the patient that does not require special verification, for example, the patient names his/her full name and date of birth, and the doctor verifies the data and photo of the patient in the medical information system. Also, the patient can provide his/her public key for identification.

The doctor makes a conclusion and prescribes a particular medicine (active substance, or INN) to the patient, and makes the appropriate EME in the patient's EMR. If the entry contains prescription medicines, the doctor performs additional identification and authentication of the patient. For complete identification and authentication, the patient chooses acceptable methods with use of a smartphone, tablet or other modern technical means (EDS, ID-Passport, ID-Card, Mobile-ID, Smart-ID, Bank-ID, FaceID, TouchID, Google-ID, Apple-ID, identification via social networks, etc., or mobile phone (via SMS). Authentication via mobile phone requires prior registration of the patient and entering his/her personal data in the information system of electronic prescriptions. In addition, an electronic document such as ID-passport, ID-Card, etc. can be used, which requires installation of additional standardized software and hardware equipment for the doctor's workplace with its connection to the relevant third-party services.

If the doctor does not have necessary technical means to read the identification media provided by the patient, another available method is chosen. Patient authentication by paper certificates is allowed only under the simplified procedure and cannot be used for prescribing of prescription and prescription medicines with special control over circulation.

If EME contains prescription medicines with special control over circulation, such as narcotic drugs, psychotropic drugs, and their precursors, the doctor performs multi-factor authentication of the patient that includes at least two available unique authentication methods. The patient passes the necessary information to the doctor and undergoes the appropriate process of identification and authentication.

At the doctor's work terminal 2 the doctor generates and sends a transaction to the server of electronic prescriptions 3. The transaction contains at least information on the patient (his identifier and/or public key), information on the prescribed medicines (identifier, name, strength, release form, quantity, daily dose, method of administration, duration of treatment, etc.), information on the doctor who performs the transaction (his/her ID and/or public key), date/time and validity period of the prescription. The transaction is signed with the doctor's private key.

The corresponding entry of a prescription signed by the doctor's private key is transmitted to the server of electronic medical entries 4, wherein the data access authorization service 5 is responsible for managing access rights to the corresponding EME. The server of electronic prescriptions 3 receives the transaction, verifies accreditation of the doctor by obtaining the doctor's public key from the database of accredited doctors 9 and verifying validity of the signature of the transaction. If the transaction is valid, the electronic prescription is stored on the server of electronic prescriptions 3.

The doctor and/or the patient receives a notification on generation of the electronic prescription with its unique electronic prescription identifier and validity period of the electronic prescription.

A unique electronic prescription identifier is a sequence of characters that can be transmitted to a patient by a doctor verbally or printed in the form of a text, barcode, or other graphic form for further automatic reading during implementation of the electronic prescription by the patient or directly by sending a message with a code and/or barcode and/or graphic code to the patient's smartphone or other means of communication, or via SMS-message.

The electronic prescription can be discharged only in the its allotted validity period. It is not possible to dispense medicines under an expired electronic prescription. The electronic prescription can be revoked by the doctor during its validity period if it has not yet been fully implemented (discharged) by the patient. The revoked prescription is not subject to discharging.

The prescription can be discharged by the patient in full by one order, or separately (partially) under different items (active substances, INN) and/or partially under the quantity of medicines per item (active substance, INN). At the same time, after each successful order of medicines by the patient, the prescription is discharged partially under the corresponding items. It is not possible to make several simultaneous orders (for example, at different points of sale) for the same unit of quantity of each item of the prescription.

Obtaining prescription medicines by the patient by means of a unique electronic prescription identifier is implemented as follows.

The patient forms an order at the patient's terminal 1 or by means of the vending machine 11.3 input device, or by means of the terminal 11 of the point of sale, or by means of the self-service terminal by using an electronic prescription, namely its unique electronic identifier, or selects medicines from the list manually.

The point of sale makes an inquiry for an electronic prescription under its unique electronic prescription identifier on the server of electronic prescriptions 3 for the possibility of its implementation. The point of sale identifies and authenticates the patient by means of the terminal of the point of sale 11 or by means of the input device. If the prescription contains medicines with special control over circulation, such as narcotic drugs, psychotropic drugs and their precursors, the multi-factor identification and authentication of the patient is performed. If the medicine is not a prescription medicine, identification and authentication of the patient is optional and is not performed.

On the basis of the prescription data, the point of sale agrees with the patient on the selected medicines, such as their list, quantity, etc., in the order. This applies to all medicines except over-the-counter medicines. The point of sale verifies the order items as to whether the prescribed medicines are approved for use in medical practice and validity term of their registration certificate in the electronic register of medicines and services 8 at the moment of implementation of the order. During receipt of the order the patient can verify authenticity of origin of the medicines by means of the patient's terminal 1 by verifying their graphic security elements on the server of validation of security elements 7. Further, from the terminal of the point of sale 11 an electronic inquiry for at least partial discharge of the electronic prescription is sent to the server of electronic prescriptions 3 and the information on the dispensed prescribed medicines is transmitted to the goods movement recording service 13.

If the patient have ordered medicines remotely, the point of sale passes the order to the delivery service. Before delivery to the patient, the order is placed in a transparent container and a separate security graphic element with opening control is sticked thereto.

The preparations in the container are placed in such a way that all the security graphic elements of each medicine are visible and accessible for reading and subsequent inspection by the patient without opening the container and breaking a separate security graphic element. The information on the container and the order is transmitted to the server of validation of security elements 7.

The delivery service identifies and authenticates the patient. Simplified authentication of the patient or internal authentication methods of the delivery service can be used during delivery of over-the counter medicines. During delivery of prescription medicines, the delivery service offers the patient to choose from the available authentication methods and confirm his/her identity, after which it passes the order to the patient.

To verify the identity of the patient, the delivery service uses:
1) the terminal of the delivery service 12 in the form of a bank terminal with the function of reading the data from the patient's electronic cards/documents, such as ID-Passport, ID-Card, Smart-ID, Bank-ID, etc., and/or
2) the terminal of the delivery service 12, such as a smartphone, tablet, laptop, computer, etc. with installed software to read the patient's data or sending an inquiry for authorization of the patient to a third-party service, such as EDS, ID-Passport, ID-Card, Mobile-ID, Smart-ID, Bank-ID, FaceID, TouchID, Google-ID, Apple-ID, SMS, Username-Password, personal identification software applications (e.g. “Diia”), identification via social networks, etc., and/or
3) the terminal of the delivery service 12 such as a smartphone, tablet, laptop, computer, etc. with software for scanning and recognizing the patient's identity documents, such as:
   Passport of a citizen
   Temporary identity card
   Certificate of permanent residence
   Temporary residence permit
   Refugee ID
   Passport of a citizen for travelling abroad
   Temporary card confirming identity of a citizen
   Stateless person identity card for travelling abroad
   Passport of a foreign citizen
   Driver's license
   Pensioner ID card
   Officer's identification certificate
   Military ID
   Police ID
   Student ID
   Migrant's card
   Diplomatic passport
   Service passport
   Crew member identity card
   Seafarer identity card
   Refugee's travel document
   Veteran identification card
   Identity document of a person requiring subsidiary protection
   Travel document of the person who has been granted subsidiary protection
   Identity card of a person in need of temporary protection
   Other
followed by check of the recognized data of the document by a courier
4) the terminal of the delivery service 12 by means of which the SMS-identification to the telephone number registered by the patient is initiated.

Multi-factor authentication of the patient is used for medicines with special control over circulation.

For multifactor authentication, the second authentication method is selected from an exclusive list (for example, it is not allowed to use patient authentication by means of paper documents having no appropriate level of protection) and guarantees error-free identification eliminating abuse and attempts of identity spoofing during identification.

After authentication of the patient, the delivery service provides the package with the order to the patient for possibility of check the security graphic elements of medicines and a separate security element of the container with the order.

In case of successful check, the delivery service gives the order to the patient, and the point of sale discharges the prescription on the server of electronic prescriptions 3. The information on release of medicines of the order is transmitted to the goods movement recording service 13.

The patient does not accept the order if the result of check of the security element is negative or a separate security graphic element on the container is breaked and does not allow its reading. In this case, the delivery service returns the order to the point of sale.

A separate embodiment of the invention is a method of automated provision of a patient in a medical institution with the prescription medicines, which includes the stages where:
by means of the work terminal of the medical staff 14, simplified authentication of the patient is performed, during this authentication the patient's identifier and/or the patient's public key and the set of data associated with the patient's EMR identifier stored on the server of electronic medical entries 4 are obtained, and by means of the access authorization service 5, access to EME stored in the patient's EMR on the server of electronic medical entries 4 is provided and a prescription of medicines to the patient is received, wherein
   if the prescribed medicines include at least one prescription medicine, then, by means of the patient identification and authentication service 6, standard authentication of the patient, including at least one unique method of authentication, is additionally performed, for which purpose at least one electronic inquiry is made from the work terminal of the medical staff 14 to the patient identification and authentication service 6 to confirm the identity of the patient, and
   if the prescribed medicines include at least one prescription medicine with special control over circulation, then multi-factor authentication of the patient, including at least two unique authentication methods, is additionally performed, for which purpose at least two electronic inquiries are made from the work terminal of the medical staff 14 to the patient identification and authentication service 6 to confirm the identity of the patient,
automated dispensing of the prescribed medicines to the patient is performed and, if necessary, by means of the patient's terminal 1 or the work terminal of the medical staff 14, authenticity of origin of the medicines is verified by verifying their graphic security elements on the server of validation of security elements 7, and
in case of the positive result of verification, administration of the prescribed medicines by the patient is ensured, wherein
   EME on administration of the prescribed medicines by the patient is made in the patient's EMR, for which purpose from the work terminal of the medical staff 14 an electronic inquiry signed by the private key of the medical staff is sent to the data access authorization service 5, the electronic inquiry including the identifier of the medical staff and/or public key of the medical staff, the patient's identifier and/or the patient's public key and the set of data associated with the patient's EMR identifier, and in case of successful authorization the permission to make changes to the patient's EMR on the server of electronic medical entries 4 is granted and EME associated with the patient's administration of prescribed medicines is made in the patient's EMR, after that
   by means of the work terminal of the medical staff 4, the information on dispensing of the prescribed medicines is transmitted to the goods movement recording service 13.

In the abovementioned embodiment of the method, the medical staff interacts with the server of electronic medical entries 4 to obtain doctor's prescriptions depending on the level of automation of the medical institution: using the work terminal of the medical staff 14 directly: smartphone, tablet, laptop, etc., or using the work terminal of medical staff 14 on a station of the medical institution: a workplace of medical staff with a stationary personal computer.

If the patient is unable to take medicines by himself, for example, the patient is unconscious, unable to make decisions on his/her own or has not reached the age of majority, etc. or instructs another person to receive medicines, the recipient of medicines under an electronic prescription can be:
“Recipient” is a representative of a minor child (parents, guardians, custodians, foster parents, parents-childcarers, adult family member, etc.), or
“Recipient” is a person who cares for a patient (family member or guardian or custodian).

Thus, the proposed methods for provision of a patient with electronic prescription and prescription medicines and the information system of electronic prescriptions implement the possibility to verify originality of each individual medicine in the order and the entire order upon its receipt by means of the server of validation of security elements, error-free identification of a person and elimination of abuse and attempts of identity spoofing during identification by means of convenient process of identification and authentication at all stages, different levels of control at the stages of prescribing and dispensing for certain groups of medicines, use of different levels of identification and authentication of a patient during dispensing of medicines, by means of standard and multi-factor authentication, control over circulation of medicines at the level of each individual primary package of the medicine by means of the goods movement recording service, control over originality of the medicines at each stage of their movement by means of the server of validation of security elements.

1. A method of automated provision of a patient with an electronic prescription for prescription medicines that includes stages where:
by means of the doctor's work terminal, simplified authentication of the patient is performed, during this authentication the patient's identifier and/or the patient's public key and the set of data associated with the identifier of the patient's electronic medical record (EMR) stored on the server of electronic medical entries are obtained,
the medicines are prescribed to the patient by means of the doctor's work terminal, wherein
if the prescribed medicines include at least one prescription medicine, then, by means of the patient identification and authentication service, standard authentication of the patient, including at least one unique authentication method, is additionally performed, for which purpose at least one electronic inquiry is made from the doctor's work terminal to the patient identification and authentication service to confirm the identity of the patient, and
if the prescribed medicines include at least one prescription medicine with special control over circulation, then multi-factor authentication of the patient, including at least two unique authentication methods, is additionally performed, for which purpose at least two electronic inquiries are made from the doctor's work terminal to the patient identification and authentication service to confirm the identity of the patient,
a transaction is generated at the doctor's work terminal to the server of electronic prescriptions, the transaction including the doctor's identifier and/or the doctor's public key, the patient's identifier and/or the patient's public key, the set of data associated with the prescribed medicines, the set of data associated with the date/time of generation of the transaction and the set of data associated with validity period of the electronic prescription, and the generated transaction is signed with at least the doctor's private key, wherein
electronic medical entry (EME) with prescribed medicines is made in the patient's EMR, for which purpose an electronic inquiry signed by the doctor's private key is sent from the doctor's work terminal to the data access authorization service, the electronic inquiry including the doctor's identifier and/or the doctor's public key, the patient's identifier and/or the patient's public key and the set of data associated with the patient's EMR identifier, and in case of successful authorization the permission to make changes to the patient's EMR on the server of electronic medical entries is granted and EME associated with the prescribed medicines is made in the patient's EMR,
the transaction signed by the doctor's private key is received on the server of electronic prescriptions and the doctor's accreditation is verified by sending an inquiry for the doctor's public key to the database of accredited doctors and verifying thereby validity of the signature of the transaction, and
in case of the positive verification of the doctor's accreditation, the electronic prescription is saved on the server of electronic prescriptions, after that
a message of generation of the electronic prescription including a unique electronic prescription identifier and its term is received from the server of electronic prescriptions and it is sent to the patient's terminal.

2. The method according to claim 1, wherein the message of generation of the electronic prescription is sent from the server of electronic prescriptions to the doctor's work terminal or the patient's terminal.

3. A method for automated receipt of prescription medicines by means of a unique electronic prescription identifier that includes the stages where:
an electronic order of the prescribed medicines is generated at the point of sale by entering a unique electronic prescription identifier into the terminal of the point of sale,
by means of the terminal of the point of sale, a transaction is generated to the server of electronic prescriptions, the transaction including a unique electronic prescription identifier, the patient's identifier and/or the patient's public key, a set of data associated with the prescribed medicines, the identifier of the point of sale and/or the public key of the point of sale, and the generated transaction is signed with at least the private key of the point of sale, wherein
if the prescribed medicines include at least one prescription medicine, then, by means of the patient identification and authentication service, standard authentication of the patient, including at least one unique authentication method, is additionally performed, for which purpose at least one electronic inquiry is made from the terminal of the point of sale to the patient identification and authentication service to confirm the identity of the patient, and
if the prescribed medicines include at least one prescription medicine with special control over circulation, then multi-factor authentication of the patient, including at least two unique authentication methods, is additionally performed, for which purpose at least two electronic inquiries are made from the terminal of the point of sale to the patient identification and authentication service to confirm the identity of the patient, after that
possibility of sale of the prescribed medicines is verified, for which purpose an electronic inquiry is made from the terminal of the point of sale to the electronic register of medicines and services and an automated response, including information on whether the prescribed medicines are approved for medical practice and validity term of their registration certificate, is received, and
validity of the transaction signed with at least the private key of the point of sale is verified on the server of electronic prescriptions, for which purpose an inquiry for the public key of the point of sale is made to the database of registered points of sale, validity period of the electronic prescription and its validity are verified and, in case of the positive result of verifications from the server of electronic prescriptions, confirmation of possibility of delivery of the order of the prescribed medicines is sent to the terminal of the point of sale, after that
automated dispensing of the ordered medicines to the patient is performed and, if necessary, by means of the patient's terminal, authenticity of origin of the medicines is verified by verifying their graphic security elements on the server of validation of security elements, wherein
by means of the terminal of the point of sale, an electronic inquiry for at least partial discharge of the electronic prescription is sent to the server of electronic prescriptions, and the information on dispensing of the prescribed medicines is transmitted to the goods movement recording service.

4. A method for automated delivery of prescription medicines to a patient by means of a unique electronic prescription identifier that includes the stages where:
an electronic order of prescribed medicines is generated at the patient's terminal, the electronic order including a unique electronic prescription identifier, the patient's identifier and/or the patient's public key, the set of data associated with the prescribed medicines, after that the electronic order is signed with the patient's private key and transmitted via the communication channels to the terminal of the point of sale,
the electronic order signed with the patient's private key is received by means of the terminal of the point of sale and a transaction is generated to the server of electronic prescriptions, the transaction including a unique electronic prescription identifier, the patient's identifier and/or the patient's public key, the set of data associated with the prescribed medicines, the identifier of the point of sale and/or the public key of the point of sale, and the generated transaction is signed with at least the private key of the point of sale, wherein
if the prescribed medicines include at least one prescription medicine, then, by means of the patient identification and authentication service, standard authentication of the patient, including at least one unique authentication method, is additionally performed, for which purpose at least one electronic inquiry is made from the terminal of the point of sale to the patient identification and authentication service to confirm the identity of the patient, and
if the prescribed medicines include at least one prescription medicine with special control over circulation, then multi-factor authentication of the patient, including at least two unique authentication methods, is additionally performed, for which purpose at least two electronic inquiries are made from the terminal of the point of sale to the patient identification and authentication service to confirm the identity of the patient, after that
possibility of sale of the prescribed medicines is verified, for which purpose an electronic inquiry is made from the terminal of the point of sale to the electronic register of medicines and services and an automated response, including information on whether the prescribed medicines are approved for medical practice and validity term of their registration certificate, is received, and
validity of the transaction signed with at least the private key of the point of sale is verified on the server of electronic prescriptions, for which purpose an inquiry for the public key of the point of sale is made to the database of registered points of sale, validity period of the electronic prescription and its validity are verified and, in case of the positive result of verifications from the server of electronic prescriptions, confirmation of possibility of delivery of the order of the prescribed medicines is sent to the terminal of the point of sale, after that
the prescribed medicines are placed in a container with opening control protected by a separate security element, wherein the container with opening control is configured to ensure verification of the graphic security elements of individual medicines therein without opening the container, the information on dispensing of the prescribed medicines is transmitted to the goods movement recording service and automated dispensing of the prescribed medicines is performed to the delivery service in the container with opening control and the container with the prescribed medicines is delivered to the patient, after that identification and authentication of the patient is performed by means of the terminal of the delivery service, wherein
if the prescribed medicines include at least one prescription medicine, then, by means of the patient identification and authentication service, standard authentication of the patient, including at least one unique authentication method, is additionally performed, for which purpose at least one electronic inquiry is made from the work terminal of the delivery service to the patient identification and authentication service to confirm the identity of the patient, and
if the prescribed medicines include at least one prescription medicine with special control over circulation, then multi-factor authentication of the patient, including at least two unique authentication methods, is additionally performed, for which purpose at least two electronic inquiries are made from the work terminal of the delivery service to the patient identification and authentication service to confirm the identity of the patient,
if necessary, by means of the patient's terminal, integrity of the separate security element of the container and authenticity of origin of the medicines are verified by verifying their graphic security elements on the server of validation of security elements, wherein verification of medicines is performed without opening the container and breaking the separate security element of the container, and, in case of the positive result of verification, the order is delivered to the patient, after that
by means of the terminal of the point of sale, an electronic inquiry for at least partial discharge of the electronic prescription is sent to the server of electronic prescriptions and the information on dispensing of the prescribed medicines is transmitted to the goods movement recording service, and
in case of breaked separate security element of the container or negative result of verification of authenticity of origin of the medicines, the container with medicines is returned to the point of sale.

5. The method according to claim 1, wherein the data associated with the prescribed medicines include the data associated with the name of the active substance and/or the international non-proprietary name (INN) of the medicine.

6. The method according to claim 1, wherein the prescription medicines with special control over circulation are narcotic drugs, psychotropic drugs or their precursors.

7. The method according to claim 1, wherein the unique authentication methods are EDS, ID-Passport, ID-Card, Mobile-ID, Smart-ID, Bank-ID, FaceID, TouchID, Google-ID, Apple-ID, identification via social networks, and SMS-authentication.

8. The method according to claim 1, wherein the data associated with the prescribed medicines include identifiers of medicines, name of medicines, information on release form, quantity, daily dose, method of administration, duration of treatment.

9. The method according to claim 3, wherein generation of the electronic order of the prescribed medicines is performed by means of the patient's terminal or the vending machine, or the work terminal of a pharmacy, or the self-service terminal.

10. The method according to the claim 9, wherein the patient's terminal is a mobile phone, or smartphone, or tablet, or laptop, or personal computer, or e-book, or other communication device.

11. The method according to claim 3, wherein the point of sale is a point of sale of medicines equipped with a terminal of a point of sale.

12. The method according to the claim 11, wherein the point of sale is a vending machine or an electronic trading platform, or a pharmacy, or an online pharmacy, or an accredited distributor of medicines.

13. The method according to claim 1, wherein the unique electronic prescription identifier is a sequence of characters or a QR code, or a bar code, or a combination thereof.

14. The method according to claim 3, wherein for verifying authenticity of origin of the medicines the self-service terminals installed at the points of sale are additionally used.

15. An information system of electronic prescriptions that implements the method according to claim 1 and that comprises;
the server of electronic prescriptions, including the database of accredited doctors and the database of points of sale, configured to process electronic transactions, store electronic prescriptions, verify accreditation of the doctors and verify the registered points of sale,
the server of electronic medical entries configured to store the patient's EMR, including a plurality of EME, and to provide access thereto,
the data access authorization service configured to control the data access rights to the data of the server of electronic medical entries, wherein the data include EMR with all EMEs,
the electronic register of medicines and services, including the database providing storage and processing of information associated with medicines, medical devices and services, configured to verify the possibility of sale of the prescribed medicines,
the patient identification and authentication service configured to identify and authenticate the patient by the patient's identifier and verify authenticity of the identification data provided by the patient, wherein the identification and authentication service is specially adapted to perform standard and multi-factor authentication of the patients,
the server of validation of security elements configured to be accessible by the patient's terminal and verify the graphic security elements and the separate security element,
the doctor's work terminal configured to generate electronic transactions, send electronic inquiries and sign them with the doctor's private key, wherein the doctor's work terminal is specially adapted to create an EMR on the server of electronic medical entries and enter/edit EME therein,
the patient's terminal configured to access the patient identification and authentication service and perform standard and multi-factor authentication of the patient, and access to the server of validation of security elements to verify the graphic security elements,
the electronic terminal of the point of sale including a communication module and a means of reading for a unique electronic prescription identifier, wherein the electronic terminal of the point of sale is configured to initiate standard and/or multi-factor authentication of the patient and dispense the prescribed medicines to the patient, and access the server of electronic prescriptions,
the terminal of the delivery service configured to initiate the procedure of identification and authentication of the patient,
wherein the patient identification and authentication service, the electronic register of medicines and services, the doctor's work terminal, the electronic terminal of the point of sale are connected to the server of electronic prescriptions, the data access authorization service is connected to the server of electronic medical entries.

16. The method according to claim 4, wherein the data associated with the prescribed medicines include at least one of the data associated with the name of the active substance or the international non-proprietary name (INN) of the medicine.

17. The method according to claim 4, wherein the prescription medicines with special control over circulation are narcotic drugs, psychotropic drugs or their precursors.

18. The method according to claim 4, wherein the unique authentication methods are EDS, ID-Passport, ID-Card, Mobile-ID, Smart-ID, Bank-ID, FaceID, TouchID, Google-ID, Apple-ID, identification via social networks, and SMS-authentication.

19. The method according to claim 4, wherein the data associated with the prescribed medicines include identifiers of medicines, name of medicines, information on release form, quantity, daily dose, method of administration, and duration of treatment.
